(19) United States
(12) Patent Application Publication
(10) Pub. No.: US 2009/0149700 A1
(43) Pub. Date: Jun. 11, 2009

(54) METHOD AND APPARATUS FOR PUBIC SLING INSERTION

(76) Inventors: RUBEN GARCIA, ATHENS, TX (US); DAVID L. NICHOLS, BROWNSBORO, TX (US); WILLIAM F. DAVIES, JR., ATHENS, TX (US)

Correspondence Address: HOWISON & ARNOTT, L.L.P, P.O. BOX 741715, DALLAS, TX, 75374-1715, US

(21) Appl. No.: 12/262,910

(22) Filed: Oct. 31, 2008

Related U.S. Application Data
(60) Provisional application No. 60/984,773, filed on Nov. 2, 2007.

(51) Int. Cl. A61F 13/00; A61B 17/122

(52) U.S. Cl. 600/37; 606/151

(57) ABSTRACT

An adjustable-length support sling assembly for internal placement within a patient includes a generally flat sling member formed of a mesh material. Each respective end of the sling member is permanently affixed to a corresponding anchor member. A portion of the sling member adjacent each respective end is folded upon itself by a predetermined length and releasably secured within an annular slot in the corresponding anchor member. The dimensions of each respective annular slot is selected to retain the corresponding folded portion of the sling member within the slot until sufficient tension is applied by the sling member that exceeds a respective predetermined tension level. The anchor members are securable in the tissue of a patient by means of the proximally-directed prongs.

### CROSS-REFERENCE TO RELATED APPLICATIONS

This application claims priority to U.S. Provisional Application Ser. No. 60/984,773, filed on Nov. 2, 2007, and entitled “METHOD AND APPARATUS FOR PUBIC SLING INSERTION,” the specification of which is incorporated herein by reference.

### TECHNICAL FIELD

This disclosure relates to surgical methods and apparatus for the internal placement of a supporting sling (tape) within in the pubic/pelvic region of female patients, and in particular, to apparatus and methods for trans-vaginal placement and post-surgical adjustment of such slings.

### BACKGROUND

It is known to treat female stress urinary incontinence (SUI) and other pubic region disorders by positioning a sling (also known as a “tape”) within the tissues of the pubic/pelvic region, e.g., to support or stabilize the urethra. A variety of techniques for such procedures have been developed and described, including retro-pubic tension-free vaginal tape (TVT), trans-obturator outside-in TVT and inside-out trans-obturator urethral suspension.

Of the existing sling placement techniques, some require multiple entry incisions for placement and tying or anchoring of the sling. Others may use a single entry incision, but require complex apparatus and procedures for placement, tying or anchoring and tensioning. A need therefore exists, for a surgical procedure and apparatus for the placement of an internal pubic/pelvic support sling that requires only a single entry incision, and further that requires simple apparatus and procedures for placement, anchoring and tension of the sling.

Regardless of the placement technique used, properly tensioning of the internal pubic/pelvic support sling is important for optimum treatment. If the sling tension is too high, the patient may be unable to empty completely or to urinate at all. Unfortunately, it is often impossible to assess whether the sling tension is correct until the patent recovers after surgery. At that point, if the sling tension is not optimal, it may be necessary to surgically re-enter the patient to adjust the tension of the sling, e.g., by relocating one or both anchors. Such surgical re-entry is very undesirable, as it may significantly increase the time and cost of the procedure and/or increases the chance of post-surgical complications. A need therefore exists, for a surgical procedure and apparatus for the placement and anchoring of an internal support sling that may be adjusted for tension by non-invasive (i.e., non-surgical) means after the patient has recovered.

During surgical placement of internal pubic/pelvic slings and anchors within the patients, it is frequently necessary for the physician to manually dissect intervening tissue (e.g., fascia, fatty tissue, etc.) using knife or scissors along the route between the incision and the anchor sites, or from one anchor site to the other. Such manual dissection makes the procedure more difficult for the physician, increases the likelihood of bruising or bleeding in the patient, and may increase the chance of inadvertent perforation of the bladder, blood vessels and other organs or tissues. A need therefore exists, for a surgical procedure and apparatus for the placement of an internal support sling that minimizes the amount of manual dissection required to allow placement and anchoring of the sling.

### SUMMARY

In one aspect thereof, an adjustable-length support sling assembly for internal placement within a patient is disclosed. An elongated, generally flat sling member formed of a mesh material having a first end and a second end is provided. A first anchor member is disposed at the first end of the sling member, and a second anchor member disposed at the second end of the sling member. Each respective anchor member has a generally tubular wall that defines a needle passageway that extends longitudinally therethrough from a proximal end to a distal end. The wall of each respective anchor member defines a plurality of proximally-directed prongs that project from the outer surface of the wall and an enlarged collar portion at the proximal end of the wall. The collar portion has a wall thickness that is substantially greater than the wall thickness at the distal end. The collar portion of each respective anchor member further defines an annular slot formed in the proximal end of the anchor member that extends longitudinally toward the distal end. The annular slot is concentrically disposed with respect to the needle passageway. Each respective end of the sling member is permanently affixed to the corresponding anchor member. A portion of the sling member adjacent each respective end is folded upon itself by a predetermined length and releasably secured within the annular slot of the corresponding anchor member. The dimensions of each respective annular slot is selected to retain the corresponding folded portion of the sling member within the slot until sufficient tension is applied by the sling member that exceeds a respective predetermined tension level. The anchor members are securable in the tissue of a patient by means of the proximally-directed prongs. Each respective anchor member will maintain tension on the sling member up to the corresponding predetermined tension level. Each respective anchor member will release the corresponding predetermined length of sling member from the corresponding annular slot when sufficient tension is applied by the sling member that exceeds the corresponding predetermined tension level.

In one embodiment, the adjustable-length support sling assembly includes a removable sheath including a cover portion and a cord portion. The cover portion is disposed over each respective anchor member and covers the plurality of proximally-directed prongs projecting from the anchor members to facilitate placement of the anchor member in a patient. One or more longitudinally extending scores may be formed in the cover portion of the removable sheath to facilitate removal of the sheath after the anchor member is placed in a patient. In one variation, the removable sheath includes a non-compliant medical balloon attached to the sheath to facilitate deployment of the sling. The non-compliant medical balloon may be attached to an exterior surface of the cover portion of the removable sheath such that the balloon extends substantially parallel to the cover portion of the sheath.

In another aspect, a surgical method for treating female urinary incontinence is disclosed. First, an incision is made in the vaginal wall. Para-urethral dissection is performed through the incision toward the minor obturator muscles. A stylet carrying a first end of an adjustable sling assembly is inserted through the incision and laterally through the dissected tissue into a first of the minor obturator muscles. The first end of the adjustable sling assembly includes a first anchor member attached to a first end of a flexible sling member. A protective sheath is removed from the first anchor member while its position is maintained in the first of the minor obturator muscles, so as to expose rearward facing prongs on the surface of the anchor. The stylet is withdrawn from the incision, leaving the first anchor member embedded in the first minor obturator muscle. The flexible sling member is then routed across the neck of the urethra. A stylet carrying a second end of the adjustable sling assembly is inserted through the incision and laterally through the dissected tissue into a second of the minor obturator muscles. The second end of the adjustable sling assembly includes a second anchor member attached to a second end of the flexible sling member. A protective sheath is removed from the second anchor member while its position is maintained in the second of the minor obturator muscles so as to expose rearward facing prongs. The stylet is withdrawn from the incision, leaving the second anchor member embedded in the second minor obturator muscle which creates tension in the flexible sling member.

In yet another aspect, a surgical apparatus for positioning a pubic sling in the endopelvic region is disclosed. The apparatus includes an elongated stylet having a distal end, a proximal end and a balloon port therebetween. The distal end forms a trochar point. A portion of the stylet between the balloon port and the proximal end is hollow. A handle is mounted to the proximal end of the stylet. A non-compliant surgical balloon is concentrically mounted over the stylet such that the balloon port is in communication with the interior of the balloon. A fluid reservoir is operatively connected to the interior of the balloon via the hollow portion of the stylet and the balloon port. A fluid compression assembly that selectively compresses the fluid in the reservoir is provided. The fluid compression assembly can alternately fill and drain the balloon to selectively inflate and deflate the balloon to dissect tissue of the endopelvic region.

In another aspect, an anchor for an adjustable-length support sling assembly for internal placement within a patient is disclosed. The anchor includes a generally tubular outer member defining a longitudinal cavity with a plurality of proximally-directed prongs projecting from an outer surface thereof. A generally tubular inner member is slidably disposed within the longitudinal cavity of the tubular outer member such that the inner member may move a limited distance relative to the tubular outer member. The inner tubular member defines a needle passageway that extends from a proximal end to a distal end of the inner member. The inner tubular member may include an enlarged end portion with an annular slot extending into the enlarged portion for receiving an end of a sling. The annular slot may be concentrically disposed with respect to the needle passageway.

A spring member, such as a coil spring, is positioned to bias the tubular inner member relative to the tubular outer member such that the tubular inner member can move a limited distance relative to the tubular outer member. Thus, when an end of the sling is permanently affixed to the tubular inner member of the anchor, the inner tubular member may still move a limited distance relative to the outer tubular member when the anchor is secured in the tissue of a patient by means of the proximally-directed prongs.

In one variation, the spring is disposed in an annular recess formed in an inner wall of the outer tubular member. The spring is constrained between a flanged end of the inner tubular member and an annular end wall of the recess. The anchor may be provided with a removable sheath having a cover portion and a cord portion. The cover portion is disposed over the anchor to cover the plurality of proximally-directed prongs projecting from the anchor member to facilitate placement of the anchor member in a patient. An elongated, non-compliant medical balloon may be attached to an exterior portion of the cover portion of the removable sheath.

### BRIEF DESCRIPTION OF THE DRAWINGS

For a more complete understanding, reference is now made to the following description taken in conjunction with the accompanying Drawings in which:

FIG. 1 is a side view of an adjustable sling assembly and associated surgical placement instruments in accordance with one aspect of the disclosure;

FIG. 2 is an expanded view of the adjustable sling assembly of FIG. 1;

FIG. 3A is an enlarged side view of the end portion of the adjustable sling assembly;

FIG. 3B is a distal end view of the end portion of the anchor member in FIG. 3A;

FIG. 3C is a cross-sectional view taken through the proximal end of the anchor member along line B-B of FIG. 3A;

FIG. 3D is a cross-section side view of the anchor member taken along line A-A of FIG. 3B;

FIG. 4 is a schematic view of the end portion of an adjustable sling assembly showing one possible configuration of folded mesh within the anchor slot;

FIGS. 5A-5F illustrate one method for forming an adjustable sling assembly in accordance with another aspect;

FIG. 6 is a side view of a tunneller apparatus for placement of the adjustable sling assembly in accordance with another aspect;

FIG. 7 is a side view of the needle portion of the tunneller apparatus of FIG. 6;

FIGS. 8A-8C are various views of the removable sheath in accordance with another aspect;

FIGS. 9-12 schematically illustrate a surgical procedure for placement and adjustment of an adjustable sling assembly in accordance with another aspect; specifically

FIG. 9 illustrates the location of the entry incision within the vagina;

FIG. 10 illustrates placement and anchoring of the first end of the adjustable sling assembly in the minor obturator muscle;

FIG. 11 illustrates placement and anchoring of the second end of the adjustable sling assembly in the opposite minor obturator muscle and routing of the sling through the endo-pelvic fascia;

FIG. 12 illustrates the suturing of the entry incision and the post-surgical non-invasive adjustment of the sling tension;

FIG. 13 illustrates surgical placement instruments having a tissue dissector in accordance with yet another aspect of the disclosure;

FIG. 14A-14C are various views of an alternate removable sheath for use with a sling assembly such as illustrated in FIG. 1; and

FIG. 15 is a lengthwise sectional view of an alternate anchor for use with a sling assembly such as illustrated in FIG. 1.

### DETAILED DESCRIPTION

Referring now to the drawings, wherein like reference numbers are used herein to designate like elements throughout, the various views and embodiments of the method and apparatus for pubic sling insertion are illustrated and described, and other possible embodiments are described. The figures are not necessarily drawn to scale, and in some instances the drawings have been exaggerated and/or simplified in places for illustrative purposes only. One of ordinary skill in the art will appreciate the many possible applications and variations based on the following examples of possible embodiments.

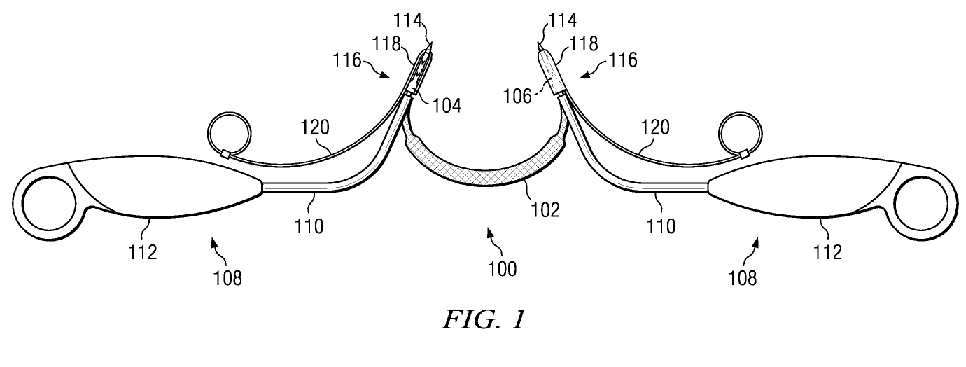

Referring to FIG. 1, there is illustrated an adjustable-length sling assembly and insertion apparatus in accordance with one aspect of the disclosure. The sling assembly 100 includes an elongated, generally flat sling member 102 having a tubular anchor member 104, 106 disposed at each end. The insertion apparatus includes one or more hand-held tunneling devices 108, each comprising a stylet 110 and a handle 112. A trochar point 114 on the end of the stylet 110 extends through the hollow center of each anchor member 104, 106. A tear-away sheath 116 including a cover 118 and a removal cord 120 is provided to cover each anchor member 104, 106. In FIG. 1, the left cover 118 is shown partially cut away to better illustrate the underlying anchor member 104.

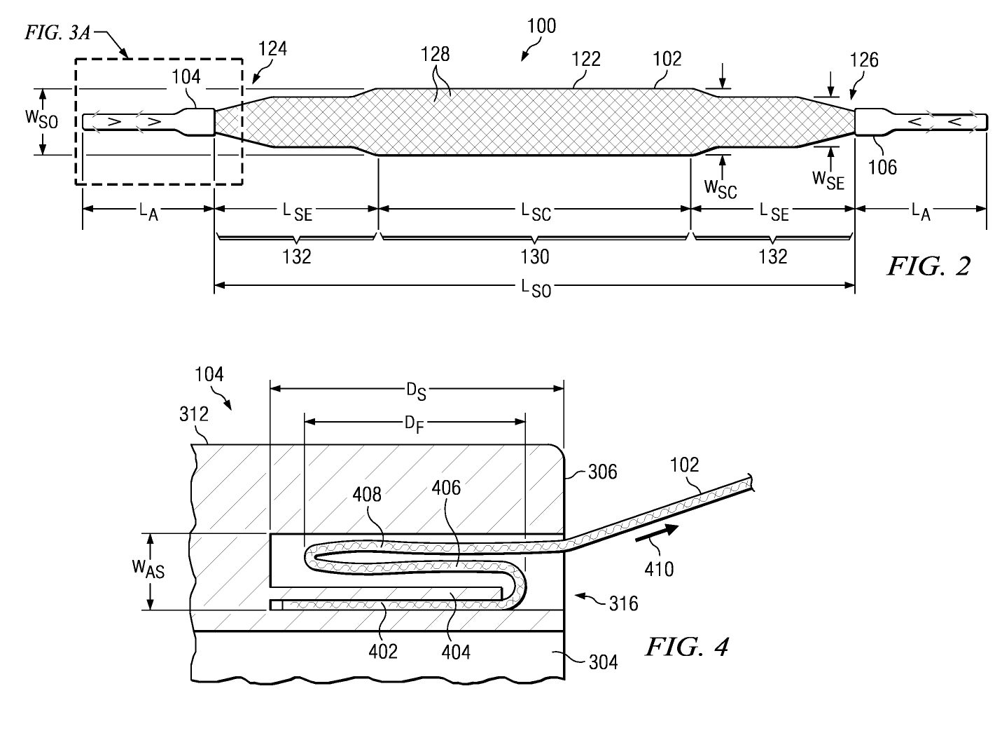

Referring now to FIG. 2 the adjustable-length sling assembly 100 is shown in further detail. The sling member 102 has an elongated body 122 with a first end 124 connected to one anchor member, e.g., member 104, and a second end 126 connected to the other anchor member, e.g., member 106. The body 122 preferably has a mesh structure, i.e., an open texture with substantially evenly-spaced holes 128. The mesh structure of body 122 may be a woven material, a knitted material, a non-woven textile or other known fabric. Alternatively, the mesh material may be a unitary sheet or film having holes 128 formed by perforation. In some embodiments, a single layer of material may be used for the sling member 102, whereas in other embodiments, multiple layers of material may be used. In one preferred embodiment, the material of the sling member 102 is polypropylene, however, other materials may be used, including, but not limited to, other medical grade polymers.

The various dimensions of the sling assembly 100 may be selected according to the parameters of the procedure to be performed, including the age, size and/or weight of the patient in addition to the planned route of the sling. In one embodiment, the anchor members 104, 106 may have a length, denoted L_{A}, in the range from about 19 mm (0.75 inches) to about 25 mm (0.98 inches) and the sling member 102 may have an overall length, denoted L_{SO}, in the range from about 90 mm (3.54 inches) to about 110 mm (4.33 inches) and an overall width, denoted W_{SO}, in the range from about 9 mm (0.35 inches) to about 11 mm (0.43 inches). In another embodiment, the sling member 102 may have a stepped or tapered configuration with a wide center section 130 and narrower end sections 132. In one stepped embodiment, the length L_{SC} of the center section 130 may be within the range from about 45 mm (1.77 inches) to about 55 mm (2.17 inches) and the width W_{SC} within the range from about 9 mm (0.35 inches) to about 11 mm (0.43 inches), while the length L_{SE} of each end section 132 may be within the range from about 22.5 mm (0.89 inches) to about 27.5 mm (1.08 inches) and the width W_{SE} within the range from about 6.8 mm (0.27 inches) to about 8.3 mm (0.32 inches). In a preferred embodiment, the center section 130 has a length L_{SC} of about 40 mm (1.57 inches) and a width W_{SC} of about 10 mm (0.39 inches).

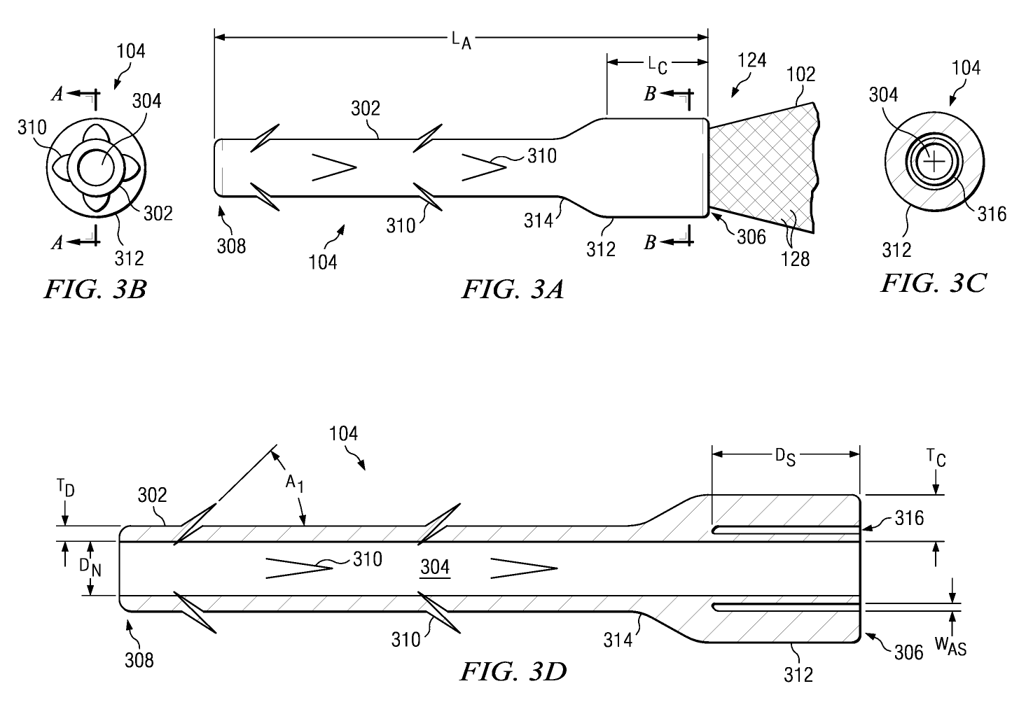

Referring now to FIGS. 3A-3D, details of the end portion of the sling assembly 100 and, particularly, of the anchor member 104 are illustrated (it being understood that the opposite anchor 106 will be substantially identical). Referring in particular to FIG. 3A, anchor member 104 is shown attached to the first end 124 of sling member 102. The anchor member 104 has a generally tubular wall 302 defining a needle passageway 304 (FIG. 3D) extending longitudinally therethrough from a proximal end 306 to a distal end 308. The wall 302 of each respective anchor member defines a plurality of proximally directed prongs 310 projecting from the outer surface thereof and an enlarged collar portion 312 at the proximal end 306. The collar portion 312 has a wall thickness T_{C} that is substantially greater than the wall thickness T_{D} at the distal end 308. For example, in one embodiment, the needle passageway 304 has a diameter of 0.058 inches and the tubular wall 302 has a wall thickness T_{D} at the distal end of 0.015 inches, yielding an overall diameter for the distal end of 0.088 inches. At the proximal end 306, the collar portion 312 has the same diameter for needle passageway 304, i.e., 0.058 inches; however, the wall thickness T_{C} is approximately 0.050 inches, yielding an overall diameter of 0.159 inches at the proximal end. In this embodiment, the prongs 310 are directed backwards, i.e., in the proximal direction, at an angle A₁ of approximately 44 degrees with respect to the tubular wall 302. The prongs 310 of this embodiment project approximately 0.028 inches above, i.e., measured radially, the outer surface of the tubular wall. Preferably, the prongs 310 will be arranged to project from the tubular wall 302 in several directions. In the embodiment shown, the prongs 310 are arranged in four opposing pairs with each pair oriented approximately perpendicular to the adjacent pairs. In other embodiments, the prongs may be arranged in identical rows, spirals or other arrangements. Preferably, the prongs 310 will not be present on the collar portion 312 of the anchor 104. Preferably, the transition between the portion of tubular wall 302 having thickness T_{D} and the collar portion 312 will include a radiused portion 314 to smooth the outer profile. In the embodiment illustrated, radiused portion 314 has a radius of approximately 0.171 inches. In typical embodiments, the length L_{C} of the collar portion 312 will be substantially less than the overall length L_{A} of the anchor member to allow sufficient room for prongs 310. In one embodiment, collar length L_{C} is less than 33 percent of anchor length L_{A}. In a preferred embodiment, collar length L_{C} is about 20 percent of anchor length L_{A}.

As best seen in FIGS. 3C and 3D, the collar portion 312 of each anchor member 104, 106 further defines an annular slot 316 formed in the proximal end 306 of the anchor member and extending longitudinally toward the distal end 308. The annular slot 316 is preferably concentrically disposed with respect to the needle passageway 304. The annular slot 316 provides a location for anchoring one end of the sling member 102. In addition, annular slot 316 provides space for the respective end of the sling to be folded upon itself by a predetermined length and releasably secured within the annular slot. As will be described further herein, the folded portion of the sling member is retained within the slot 316 until the tension applied by the sling member 102 exceeds a predetermined tension level. When the predetermined tension level is exceeded, the folded portion of the sling member is pulled from within the slot, thereby allowing adjustment of the overall sling length. In one embodiment wherein the needle passageway 304 has a diameter D_{N} of 0.058 inches and the collar portion 312 has a diameter of about 0.159 inches, the annular slot 316 has a inside diameter of 0.072 inches and an outside diameter of 0.086 inches, corresponding to an annular slot width of approximately 0.007 inches. As best seen in FIG. 3D, the annular slot 316 has a depth D_{S}. In one embodiment, slot 316 has a depth D_{S} of approximately 0.175 inches measured from the face at distal end 306 towards the proximal end 308.

Referring now to FIG. 4, there is illustrated a schematic diagram showing how the end portion of sling member 102 is secured within the annular slot 316 of the anchor member 104 (or 106) and folded upon itself to make an extendable adjustment loop. It will be noted that the radial dimension is exaggerated in FIG. 4 to clarify the structure for purposes of illustration; sling member 102 will actually fit very tightly in annular slot 316.

The outer most portion of sling member 102 includes three adjacent sections, namely secured section 402, inward fold section 406 and outward fold section 408, each section having a proximal end and a distal end. Sections 402, 406, and 408 are initially disposed within the annular slot 316. The outer most section, secured section 402, is permanently secured to an interior wall of the annular slot 316 by a securing overlay 404. The securing overlay 404 is preferably an integral part of the anchor member 104. Once it passes the proximal end of the securing overlay 404, the secured section 402 folds back in the distal direction to become the inward fold section 406. The inward fold section 406 continues in the distal direction until it approaches the distal end of the annular slot 316, whereupon the inward fold section 406 folds back on itself once again (heading back in the proximal direction) to become the outward fold section 408. The outward fold section 408 continues in the proximal direction until it exits the annular slot 316 and becomes exterior portion of the sling member 102. The distance between the distal and proximal ends of the inward fold section 406 defines the depth of fold D_{F}. D_{F} is preferably a significant portion of slot depth D_{S}.

As previously described, the secured section 402 is permanently secured within the annular slot 316 and the inward and outward fold sections 406 and 408, respectively, are tightly fitted, but not permanently secured within the annular slot. When tension is applied to the sling member 102 in the direction indicated by arrow 410, the tightness of the annular slot 316 will retain the folded sections 406 and 408 within the slot until the tension applied by the sling member exceeds a predetermined tension level. The predetermined tension level may be the same for both anchors 104 and 106 or it may be different for each anchor. When the tension applied by the sling member 102 exceeds the respective predetermined tension level of an anchor, the corresponding folded sections 406 and 408 will be pulled from the slot 316, in whole or in part, to increase the length of sling member 102 by a length up to two times D_{F} (per anchor). Thus if the folded sections are deployed from both anchor members 104, 106, the length of the sling member may be increased by four times D_{F}. In some embodiments, D_{F} is in the range of 0.2 to 0.3 centimeters, and preferably about 0.25 centimeters. This provides an extra 0.042 to 0.6 centimeters of sling length from each anchor. Preferably, about 0.5 centimeters adjustment is available from each anchor. Once the folded portions 406, 408 are withdrawn from the slot 316, the sling member will remain securely attached to anchors 104, 106 by means of the secured section 402. It is believed that by varying selecting the width of the annular slot (W_{AS}), a wide array of predetermined tensions can be achieved.

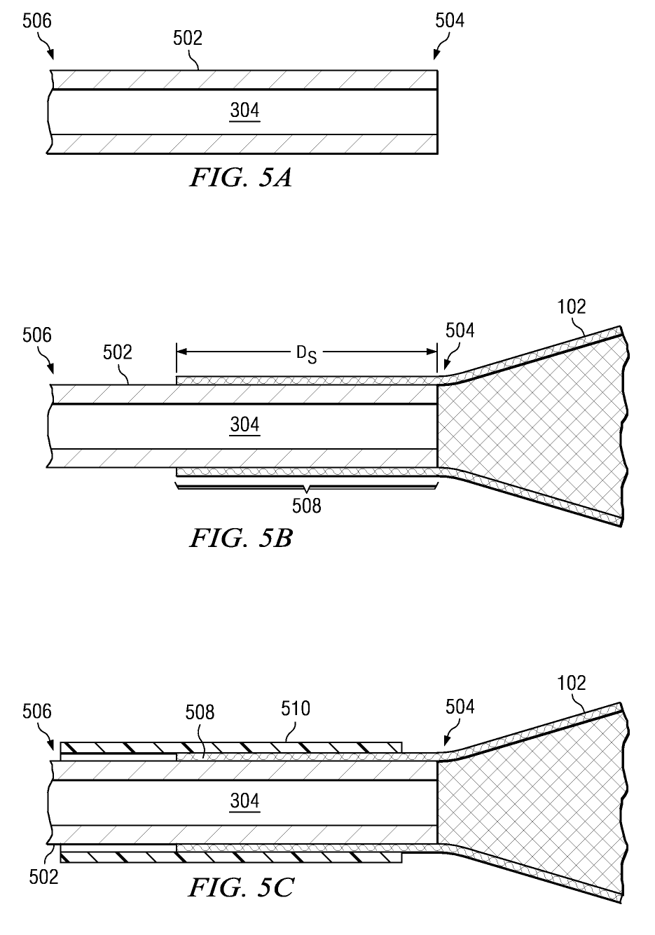

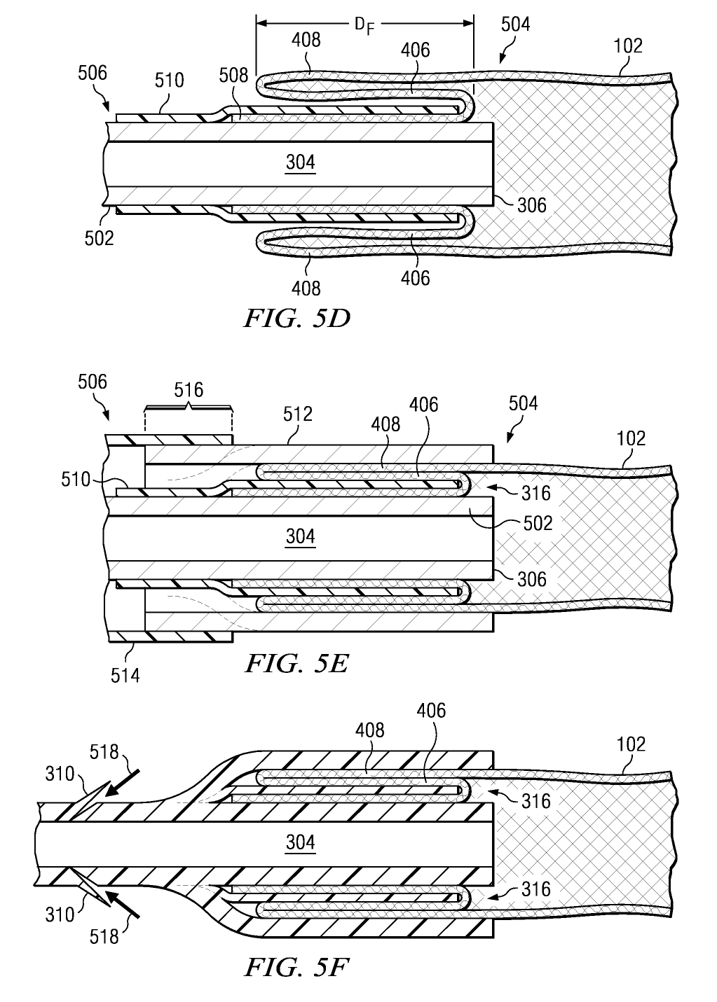

Referring now to FIGS. 5A-5F, there is illustrated one method for forming an adjustable sling assembly in accordance with one aspect. Referring first to FIG. 5A, the process begins by providing a base tube 502 having a proximal end 504 and a distal end 506. The base tube 502 will become the anchor member 104, 106. The interior passage of base tube 502 forms the inner portion of the needle passageway 304. In one embodiment, the base tube 502 is formed of polypropylene. Other plastics, resins and polymer materials may be used in other embodiments.

Referring now to FIG. 5B, the outer end 508 of the sling member 102 is positioned around the proximal end 504 of base tube 502 to a depth of approximately D_{S}. Outer end 508 will become the secured section 402 as the process continues. The outer end 508 may be secured in place around the tube either by mechanical means or by permanent or temporary adhesives. Preferably, permanent adhesives are not used because a glue joint represents a potential point of failure in the finished structure. Instead, it is preferred to use thermal bonding, i.e., welding, to attach outer end 508 to base tube 502 as described further below. Accordingly, the material of sling 102 is preferably thermally bondable to the material of base tube 502. In one embodiment, the sling member 102 is made of polypropylene and the base tube 502 is made of polypropylene.

Referring now to FIG. 5C, a first heat-shrink sleeve 510 is positioned over the base tube 502 and outer end 508 of the sling member. This first heat-shrink sleeve 510 will become the securing overlay 404 of FIG. 4. In some embodiments, heat-shrink sleeve 510 may be applied primarily to the portion of tube 502 already covered by sling end portion 508. In other embodiments, heat-shrink sleeve 510 may cover additional portions of the base tube 502 or even the entire base tube. After positioning, heat is applied to sleeve 510 causing it to shrink tightly around base tube 502 and sling end portion 508. During the heat-shrink process, sleeve 510 will exert significant pressure against sling portion 508 and the base tube 502. Preferably, first heat-shrink sleeve 510 may be formed of a material thermally bondable with material of the base tube 502 and/or the material of the sling 102. The combination of head and pressure during the heat-shrink process will cause thermal bonding of compatible materials. After the heat-shrink process is complete, the outer sling end 508 will be permanently secured to the base tube 502 and/or heat-shrink sleeve 510, and preferably, may be thermally bonded to one or both of them.

Referring now to FIG. 5D, the proximal portion of the now securely attached sling section 508 is folded, first in the distal direction for a distance of approximately D_{F}, and then back in the proximal direction until it passes the proximal end 306 of tube 502. These folded portions will become the inward fold section 406 and the outward fold section 408 shown in FIG. 4.

Referring now to FIG. 5E, a pocket member 512 is positioned over the folded portions 406 and 408 and at least a portion of the remaining base tube 502. The pocket member 512 will form the outer wall of the annular slot 316. Preferably, pocket member 512 is formed of a material that is thermally bondable to the material of the base tube 502 or first heat-shrink sleeve 510, whichever is exposed on the surface. A second heat-shrink sleeve 514 is positioned over the distal end of pocket member 512 and some or all of the remaining base tube 502. When heat is applied to the assembly, second heat-shrink sleeve 514 will shrink, exerting considerable pressure against the distal end 516 of pocket member 512 and causing it to deform (as indicated by the dotted lines) as it is pressed tightly against the underlying base tube 502 and/or first sleeve 510. This application of heat and pressure will cause pocket member 512 to be permanently secured to the base tube, preferably by thermal bonding. Because second heat-shrink sleeve 514 was only located over the distal end 516 of pocket member 512, and not over the portions of the pocket member that overlie folded sections 406 and 408 of the sling, no thermal bonding occurs between the pocket member and the folded sling. The heat may cause pocket member 512 to shrink somewhat, thus tightly gripping the folded sling; however, since no thermal bonding occurs, the folded sections 406 and 408 of the sling can be pulled away from pocket member 512 when sufficient tension is applied. In contrast, since the secured section 402 is thermally bonded to the base tube 502 and/or securing overlay 404, it cannot be pulled away from the anchor member.

Referring now to FIG. 5F, after the final heat-shrink process any unbonded heat-shrink material may be removed, leaving a semi-finished anchor member with folded sling material disposed in the annular slot 316. In preferred embodiments, wherein the material of the first heat-shrink sleeve 510 and the pocket member 512 are both thermally bondable to the material of the base tube 502, the completed anchor will be a one-piece, continuous structure. At this point, the distal end of the tubing may be scythed to form prongs 310 by cutting the tubing in the direction shown by arrow 518.

In some embodiments, the anchor members 104 and 106 may act as a drug-delivery system in addition to physically securing the sling member to the patient's tissue. The anchor members, or component parts thereof, provide a way to place medication into the patient's tissues before, during and/or after deploying the anchors. In some embodiments, the anchor members may be coated with a drug, which is released into the patient after implantation. In other embodiments, the anchor members may be formed from a permeable material that is infused with a suitable drug. In still other embodiments, a capsule or other small container of drugs may be positioned inside the anchor member assembly, e.g., under sheath, so that it will be released upon deployment of the anchor. Suitable drugs include hormones, pain relievers, anti-rejection agents, and anti-inflammatory agents. The drugs may be fast-acting, extended-release or delayed-acting.

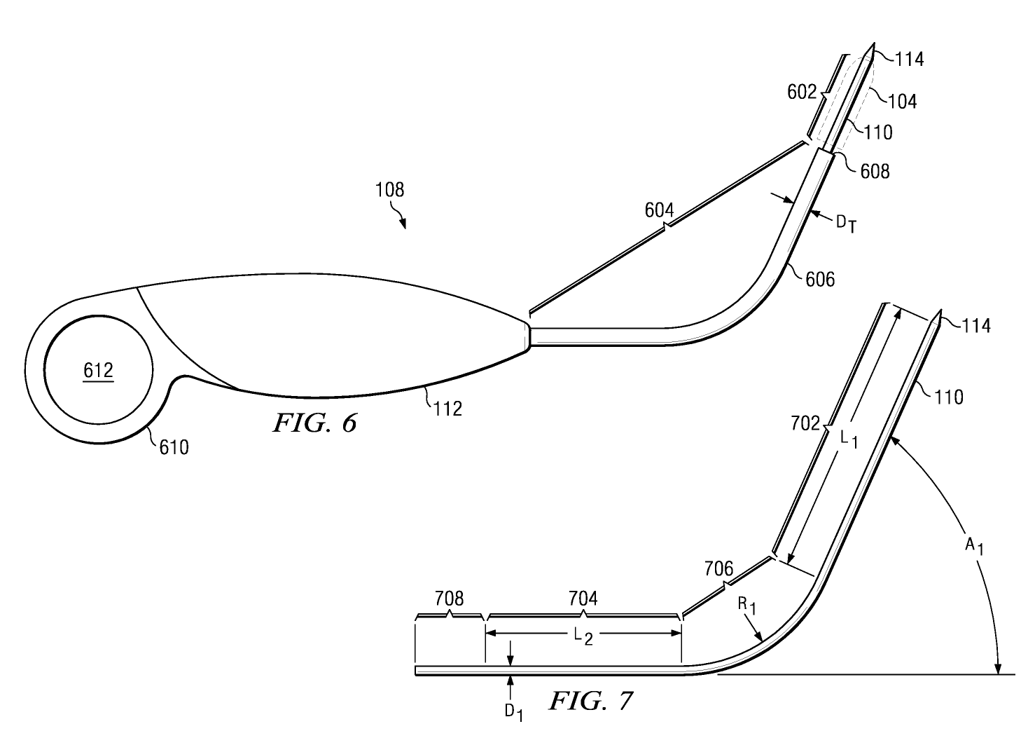

Referring now to FIG. 6, there is illustrated a tunneling device for placement of the adjustable sling assembly in accordance with another aspect. Tunneling device 108 includes a stylet 110 affixed in a handle 112. Stylet 110 includes an exposed portion 602 and a covered portion 604. Exposed portion 602 terminates in a trochar point 114. The length of exposed portion 602, not including trochar 114, generally corresponds to the length L_{A} of the anchor member 104, 106 (shown in dotted line) such that the trochar is exposed when the anchor is installed on stylet. A piece of tubing 606 is placed over the covered portion 604 of the stylet 110. The distal end 608 of the tubing 606 serves as a stop, which prevents further rearward movement of the anchor 104, 106 on the stylet during placement. The diameter D_{T} of the tubing 606 of covered portion 604 is preferably selected to be slightly larger than the diameter of the anchor, or substantially equal to the diameter of the sheath cover 118. The handle 112 is designed to be securely gripped by the surgeon. A finger grip 610 defining a finger hole 612 may be provided.

Referring now to FIG. 7, there is illustrated the stylet 110 removed from the handle. Stylet 110 is preferably made of a surgical grade stainless steel. The stylet 110 includes first and second straight portions 702 and 704 connected by a curved portion 706. First straight portion 702 preferably has a length L₁ (including trochar 114) in the range from about 2 to about 2.25 inches. In preferred embodiments, length L₁ is approximately 2.142 inches. Second straight portion 704 preferably has a length L₂ (extending from the handle) between 1.0 and 1.25 inches. In a preferred embodiment, length L₂ is approximately 1.13 inches. An embedded portion 708 may extend from the portion 704 for mounting in the handle 112. The curved portion 706 has a relatively large radius so as to move smoothly through the tissue during surgery. Curved portion 706 preferably has a radius R₁ within the range of 1.0 to 1.25 inches and more preferably, it has a radius of about 1.096 inches. Insertion of the curved portion 706 between the straight portions 702 and 704 results in an angle being formed between the two straight portions. This angle A₁ facilitates insertion of tunneling device through the initial incision and into the target areas of the body. In one embodiment, angle A₁ is within a range of 60 to 70 degrees, and in a preferred embodiment it is about 66 degrees. Stylet 110 preferably has a diameter D₁ selected to fit through the needle passageway 304 of the anchor member. In a preferred embodiment, diameter D₁ in the range from about 0.062 to about 0.054 inches, and in a preferred embodiment, diameter D₁ will be about 0.058 inches.

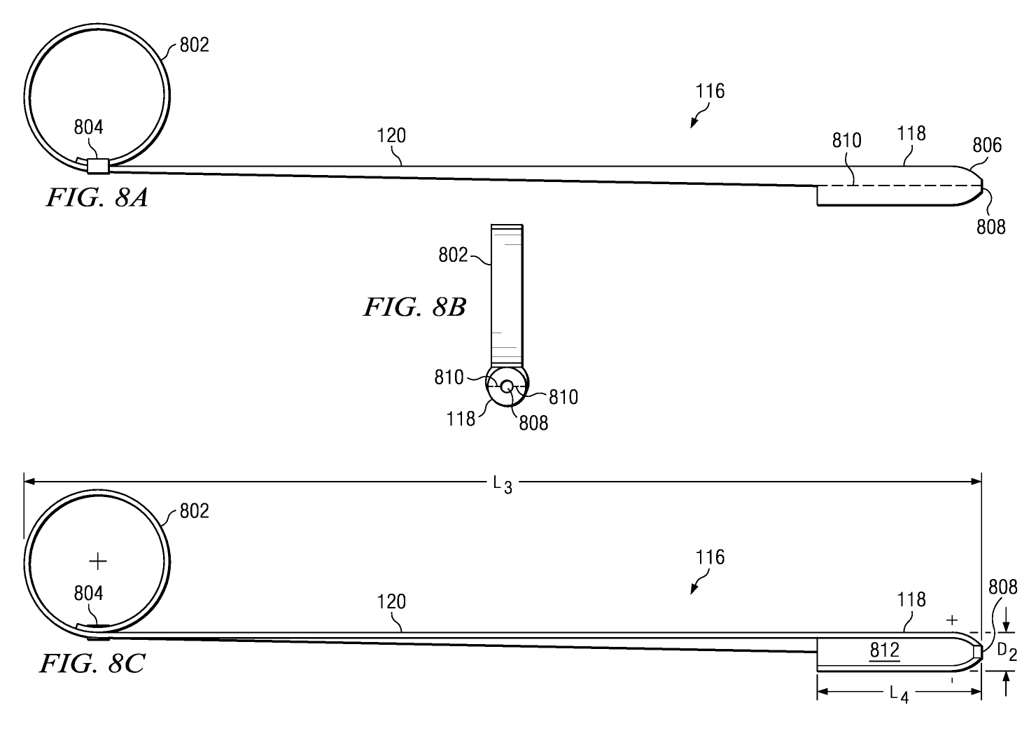

Referring now to FIGS. 8A-8C, further details of the removable sheath that covers the anchor members during installation are shown. Referring first FIGS. 8A and 8B, exterior side and front views are shown. The sheath 116 includes a cover portion 118 and a cord portion 120. Preferably, the end of the cord portion 120 will be formed into a handle 802 to facilitate removal of the sheath from the anchor. In the embodiment shown, the rear portion of cord 120 is curved into a loop and secured with a clip 804 to form a circular handle 802. The distal end of the cover 118 will preferable have a rounded profile 806 to facilitate insertion through the tissues of the patient. A hole 808 is provided on the end of the cover allowing the trochar point 114 (not shown) to protrude. A series of longitudinal scores 810 are preferably formed down the length of the cover 118 to facilitate removal of the sheath from the anchor when proper placement has been achieved.

Referring now to FIG. 8C, a cross-sectional view of sheath 116 is shown. The cover portion 118 defines an interior cavity 812 within which the anchor member is disposed during insertion. Preferably, the sheath 116 is formed from a single piece of tubing having a thermally formed end profile 806 and a cord 120 and handle 802 formed from split portions of the tubing. In one preferred embodiment, the entire sheath 116 (less clip 804) is formed from a single piece of polypropylene tubing having a wall thickness of about 0.02 inches. The overall length L₃ ranges from about 4.5 to about 5.0 inches, and in the preferred embodiment, length L₃ is about 4.85 inches. The cover portion 118 has a length L₄ sufficient to cover the anchor while leaving the trochar exposed. L₄ ranges in length corresponding to the L_{A}, but generally, L₄ will be within the range of about 0.81 to about 0.84 inches. In a preferred embodiment, L₄ is about 0.825 inches. The sheath has a diameter D₂ sufficient to cover the anchor member. In some embodiments, D₂ will be between 0.20 and 0.172 inches, but in the preferred embodiment, D₂ is 0.186 inches.

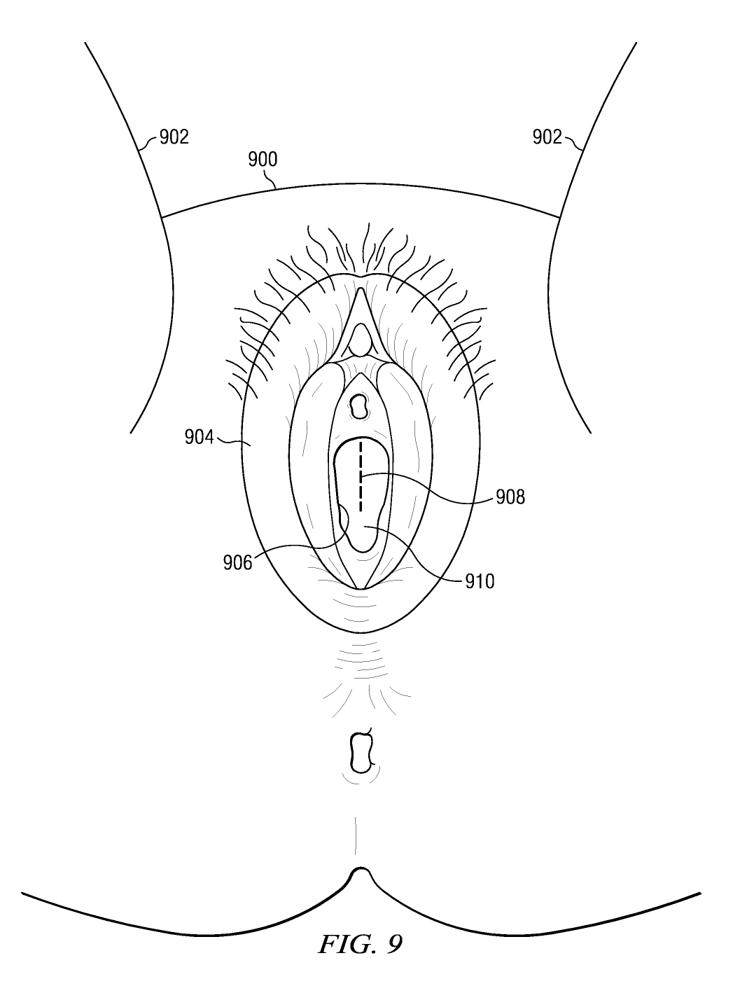

Referring now to FIGS. 9-12, a surgical procedure in accordance with another embodiment is shown. Referring first to FIG. 9, the patient 900 is first placed in the gynecological position, with legs or feet in stirrups and thighs 902 in hyperflexion. The operative field is cleaned with a standard antiseptic agent and draped. Labia minor 904 are suspended by retractors, fixation to the skin with suture or other means in order to expose the vaginal opening 906. The incision location 908 (denoted by dotted line) is shown on the anterior vaginal wall 910.

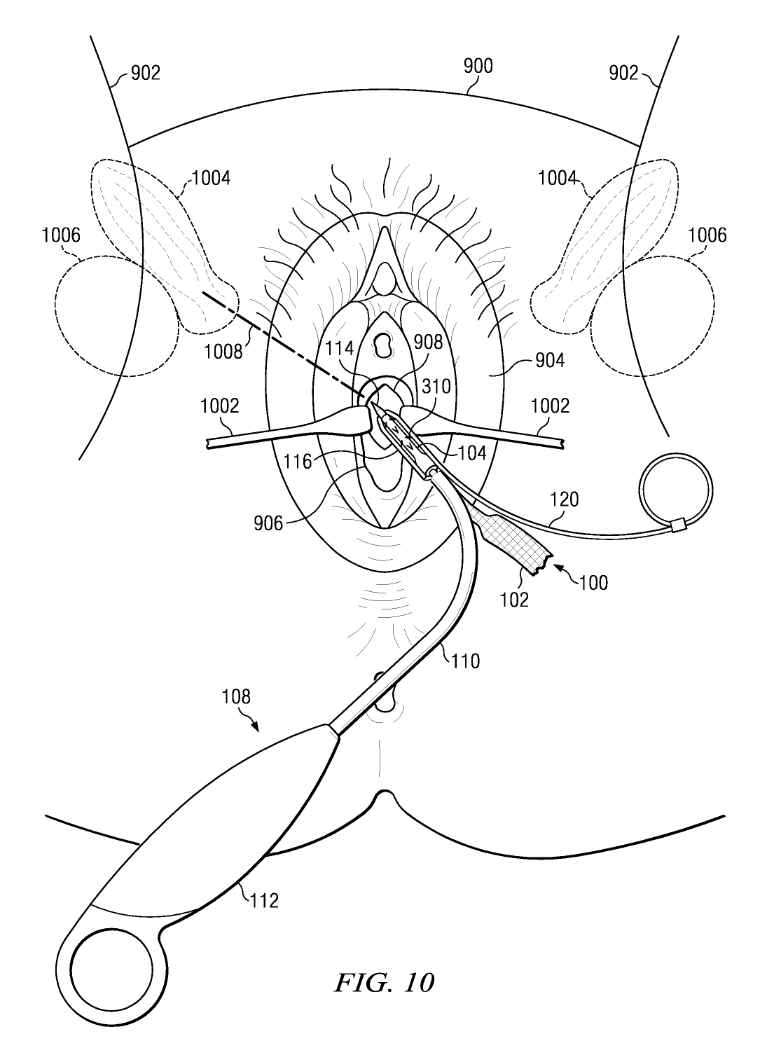

Referring to FIG. 10, tissue clamps 1002 are attached to the margins of the incision 908 to expose the underlying tissues. Para-urethral sub-vaginal dissection is then performed laterally through the incision using knife and/or scissors to a point near the minor obturator muscle 1004. This dissection creates a channel within which the flexible sling may be deployed. For reference purposes, the obturator foramens 1006 is also shown. Using the tunneling device 108, a first end of an adjustable sling assembly 100 is placed in the minor obturator muscle 1004 as follows. First, the stylet 110 with anchor member 104 and sheath 116 (shown partially broken away for purposes of illustration) is introduced through the incision 908 along the dissected sub-vaginal path (denoted by dotted line 1008). The trochar point 114 is then used to penetrate the remaining undissected tissue to reach the obturator muscle 1004. The sheath 116 covers the anchor member 104 (except exposed trochar 114) to prevent the prongs 310 from prematurely engaging the patient's tissue. This allows the surgeon to move the anchor 104 back and forth within the tissue using the handles 112 until the optimum placement is achieved. Once the anchor 104 is properly placed, the surgeon grips handle 112 firmly and then removes the sheath 116 by pulling on cord 120. Pulling cord 120 causes the cover 118 to split along scores 810 allowing the cover to pass over the stylet 110 and the anchor 104 so that it may be withdrawn from the patient 900. The rearward facing prongs 310 will now be exposed and will embed in the patient's tissue to prevent the anchor member 104 from being withdrawn and to secure the first end of the sling member 102 within the patient 900.

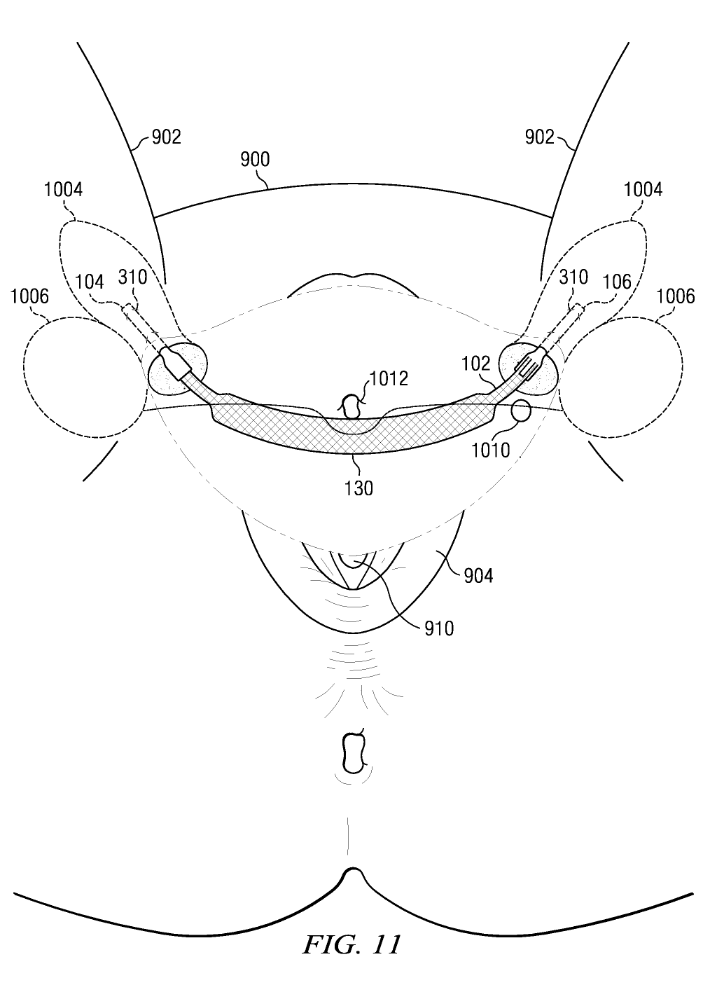

Referring to FIG. 11, selected portions of the patient's exterior tissue are omitted for purposes of illustration to show the underlying structures. Following placement of the first anchor 104, the flexible sling is deployed (i.e., laid out flat) within the dissected channel between the minor obturator muscles 1004 to cradle the mouth of the bladder. The anchor placement process is then repeated on the other side of the patient 900 to route the second anchor 106 through the endopelvic fascia 1010 and into the opposite minor obturator muscle 1004. After placement, the protective sheath 116 is removed by pulling cord 120 to expose the anchor member 106 and embed the prongs 310 in the patient's tissue. When properly placed, the central portion 130 of the sling member 102 cradles the mouth of the bladder or urethra 1012 to minimize movement during stress. The portion of the sling member 102 contacting the urethra/bladder should be substantially flat to avoid pressure-induced erosion.

The previous description presents one possible procedure for implanting the anchors 104 and 106 into the patient's tissue so as to support the sling member 102. It will be appreciated that physicians may develop alternative procedures for utilizing the device of the current invention to implant the anchors. This would include alternate incision locations, alternate dissection paths and alternate target tissues for anchor implantation. For example, in an alternative procedure, the anchors may be used to attach the sling member directly into the pelvic musculature without having to go through the obturator space when performing an anterior repair.

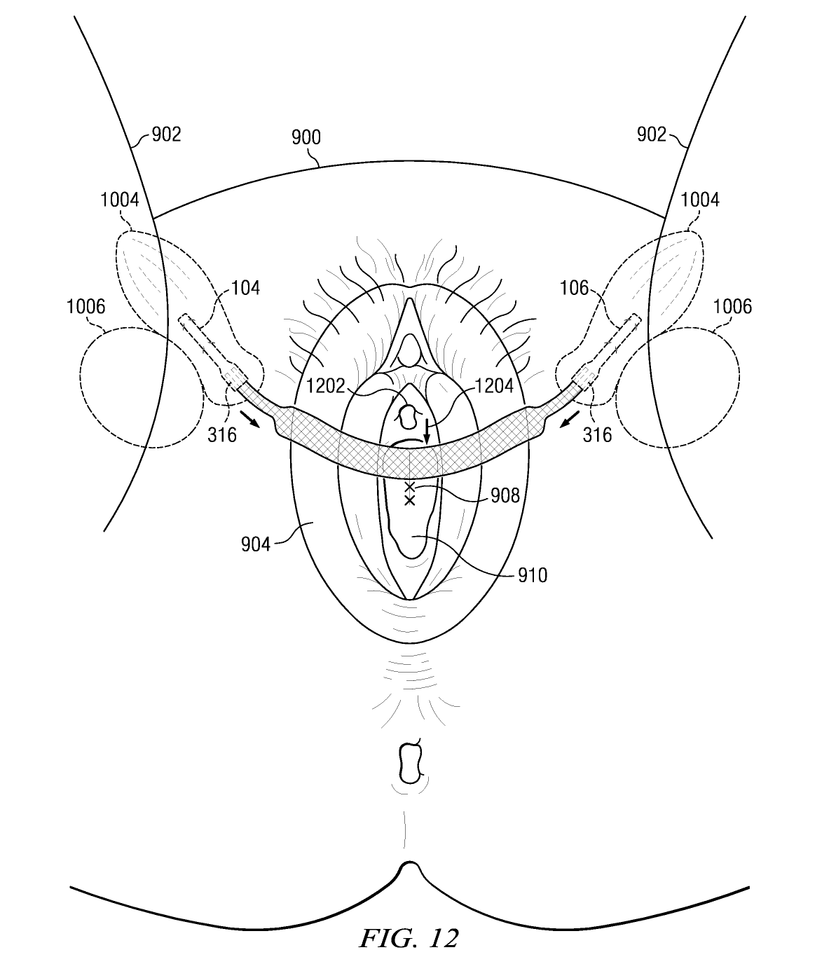

Referring to FIG. 12, post-surgical adjustment of the sling assembly is shown. After placement of the anchors 104 and 106 in the minor obturator muscles 1004 to secure the ends of the sling member 102, the surgical instruments are withdrawn and the incision 908 is sutured closed. After a suitable recovery period, the patient's bladder will fill. If the tension of the sling member is satisfactory, then the patient 900 will be able to urinate on her own. However, if the tension of the sling member is too high, then the urethra will be held too high and the patient will be unable to urinate. In prior art procedures, the latter situation would necessitate the surgeon to surgically re-enter the patient in order to adjust the tension of the sling (e.g., by re-tying the sling or relocating the anchors) to allow the patient to pass water. The device of this disclosure, however, is adjustable for length (and, thus, for tension) through non-invasive means as follows. If the sling member 102 is too tight (e.g., the patient cannot urinate), the doctor may adjust the tension by inserting a urethral dilator or sound (not shown) into the urethra 1202 into the area adjacent to the sling. The doctor then uses the urethral sound to push down on the urethra 1202 and, hence, also on the sling 102 in the direction shown by arrow 1204. Pushing against the sling 102 increases the tension applied at the anchors 104 and 106. When the tension applied by the sling 102 exceeds a predetermined level, the folded sling sections 406 and 408 will be pulled from the annular slot 316 at the end of one or both of the anchor members 104, 106. Pulling the folded portions 406 and 408 from the annular slot 316 increases the length L_{SO} of the sling by two times the folded length D_{F} for each anchor deployed. This decreases the tension of the sling enough to allow the patient to urinate without requiring surgical re-entry.

While the apparatus and procedures disclosed above are a great improvement over the prior art, the disclosed procedure still involves an incision of 1-1.5 cm in the vaginal wall. Further, it still involves significant manual dissection of the endopelvic fascia. In additional embodiments disclosed below, an apparatus and procedures for placing a flexible sling are described which require significantly smaller opening be made in the vaginal wall and significantly less manual dissection of the endopelvic fascia.

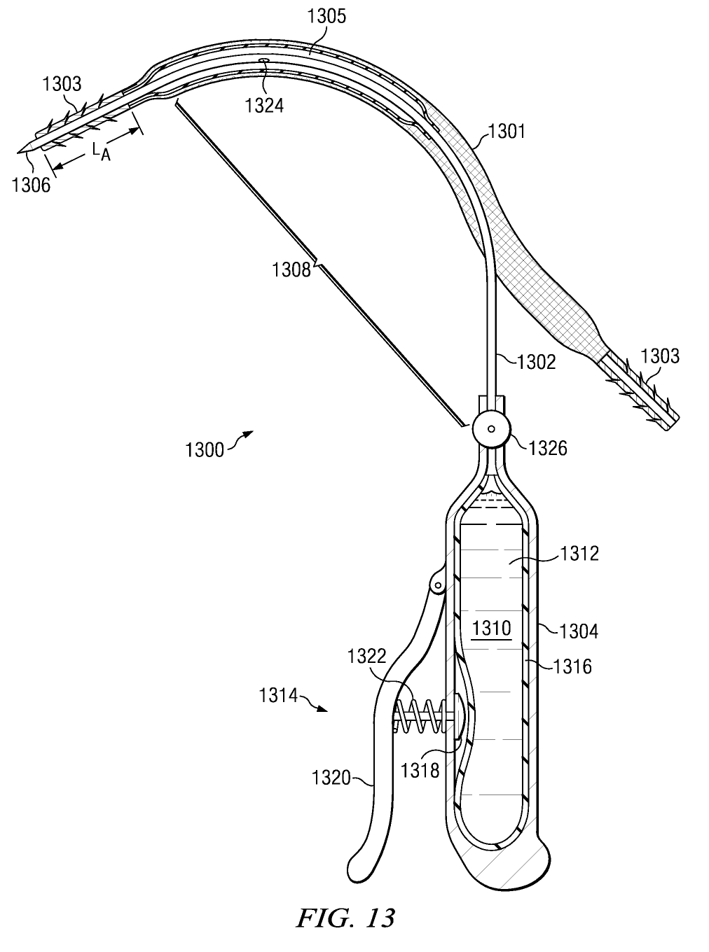

Referring to FIG. 13, an alternative tunneling device having an inflatable dissector in accordance with another aspect of the disclosure is shown. This tunneling device may be used with an adjustable sling assembly such as sling assembly 100 previously described, or it may be used with a sling assembly having a flexible sling 1301 and pronged anchors 1303 such as those shown in FIG. 13. Tunneling device 1300 comprises a stylet 1302, a handle 1304 and a dissection balloon 1305 positioned concentrically around the stylet. The stylet 1302 includes a trochar 1306 and a curved or angled profile which may be similar to that of stylet 110 shown in FIG. 7. However, stylet 1302 further includes a hollow portion 1308 extending from the handle 1304 to at least the dissection balloon 1305. A fluid reservoir 1310 is connected to the hollow portion 1308 of the stylet 1302. In the embodiment of FIG. 13, fluid reservoir 1310 may be disposed within the handle 1304, and in other embodiments it may be separate from the handle. Fluid reservoir 1310 is filled with a fluid 1312 which is preferably an incompressible fluid such as saline solution, but may be a compressible fluid such as air.

A fluid compression assembly 1314 is provided on fluid reservoir 1310 to allow selective pressurization of the fluid 1312. In the embodiment shown in FIG. 13, fluid compression assembly 1314 includes a flexible bladder 1316 lining fluid reservoir 1310, a plunger 1318 for compressing the bladder, a lever 1320 pivotally connected to the handle and a return spring 1322. Squeezing lever 1320 causes plunger 1318 to deform bladder 1316, thereby compressing fluid 1312 and causing it to flow out of reservoir 1310 through the hollow portion 1308 of the stylet 1302 and out of a port 1324 disposed inside the dissection balloon 1305. The fluid flowing from port 1324 into dissection balloon 1305 causes it to inflate. As balloon 1305 inflates, it forces apart the surrounding tissue thereby creating a channel for placement of the flexible sling. Balloon 1305 is preferably a non-compliant balloon of the type known for use in angioplasty, orthopedics, and urology. In particular, non-compliant balloons such as those described in U.S. Pat. No. 6,748,425 to Beckham and co-pending pending publication Nos. US 2006-0085022 A1 to Hayes, US 2006-0085023 A1 to Davies, Jr., and US 2006-0085024 A1 to Pepper, the disclosures of which are hereby incorporated herein by reference, may be suitable for use.

In one embodiment, dissection balloon 1305 is a non-compliant balloon having an outside diameter within a range of about 0.085 inches to about 0.095 inches, preferably about 0.089 inches, when deflated (i.e., folded), and having a diameter of a range of about 10 mm to about 15 mm, preferably about 12 mm, when inflated. The balloon 1305 will be positioned on stylet 1302 at a distance approximately L_{A} below the trochar tip 1306, L_{A} being the length of the anchor member. Balloon 1305 is sealed to the stylet 1302 at either end. In some embodiments, the length of the balloon will be within the range of about 25 mm to about 45 mm, and preferably between about 30 mm to about 40 mm. A valve 1326 maybe provided to selectively control the flow of fluid 1312 in and out of the balloon 1305. In one embodiment, valve 1326 may be a one-way valve which allows fluid to freely flow from the reservoir 1310 into balloon 1305, but only allows fluid to flow out the balloon when the valve is manually activated. It will be appreciated that other fluid compression devices known for use in cardiology and angioplasty procedures may be substituted for fluid compression assembly 1314 shown in FIG. 13.

The surgical placement of a flexible sling member using the dissecting tunneling device 1300 is similar in many respects to that previously described in association with FIGS. 9-12. The patient is first prepped as shown in FIG. 9. However, rather than creating an incision in the vaginal wall, a 2 mm to 3 mm puncture opening is created using an appropriate surgical instrument. Tunneling device 1300 may then be used to introduce a flexible sling assembly through the initial puncture into the patient's endopelvic fascia. The trochar point 1306 will create a sufficient channel through the tissue to allow pronged anchors 1303, deflated balloon 1035 and flexible sling 1301 to follow. It will be appreciated that this small channel will create less tissue damage and bleeding than procedures using manual dissection using knife or scissors.

The stylet 1302 of the tunneling device is routed through the tissue to such a point that the balloon 1305 lies in the preferred location for the sling. The dissection balloon 1305 is then inflated using fluid compression assembly 1314. Inflation of the balloon 1305 separates the surrounding tissue to create a channel suitable for deployment of the flexible sling 1301. In addition, should significant bleeding occur, the inflated balloon 1305 may be held in place for a period of time to effect tampenade. Once any tampenade and the channel dissection is completed, the balloon 1305 is deflated, e.g., using valve 1326, to allow for easier progression to the next inflation point along the desired sling channel route. The surgeon will then advance the trochar point 1306 further along the path towards the obturator muscle and repeatedly inflate and deflate the balloon 1305 to create the dissected channel. Once the trochar point 1306 reaches the target position in the obturator muscle, the anchor 1303 is deployed. If the sling assembly has adjustable anchors, e.g., anchors 104, 106, such as that described in FIG. 1, then the anchor will be deployed by removing the sheath, e.g., sheath 116, to expose the prongs and embed them in the patient's tissue before withdrawing the stylet from the patient. If using bare pronged anchors 1303, such as those shown in FIG. 13, the anchors will merely remain in place as the stylet 1302 and attached deflated balloon 1305 are withdrawn from the path and the patient.

Following placement of the first anchor, the flexible sling 1301 is deployed, i.e., laid out flat, within the dissected channel across the mouth of the bladder. The anchor placement process is repeated on the other side of the patient using balloon dissection as necessary to route the second anchor through the endopelvic fascia and into the opposite minor obturator muscle. As before, the anchor is embedded by removing a protective sheath, if present, and the balloon is deflated and the device is withdrawn from the patient. If necessary, the tunneling device 1300 and/or other dissection tools may be utilized to create any remaining channel and/or to position the flexible sling 1301 within the dissected channel so that it properly cradles the mouth of the bladder. All of these procedures are preferably accomplished through the single 2 mm to 3 mm puncture opening. After sling placement, the puncture in the vaginal wall is sutured closed. This completes placement of the sling assembly. If an adjustable anchor assembly, such as assembly 100 of FIG. 1, is used, then the tension of the sling may be adjusted (post recovery) by non-invasive means as previously described.

In another embodiment (not shown), a sling deployment balloon may be provided on the insertion apparatus to facilitate deploying the sling member within the body. The insertion apparatus may be substantially similar in most respects to the tunneling device 1300 previously discussed. However, the balloon of this alternative device will be substantially larger when inflated than the dissection balloon 1305. Rather than being inflated within dense tissue for dissection purposes, the sling deployment balloon may be inflated within internal cavities to expand the net and allow it to be positioned in a more evenly-distributed manner.

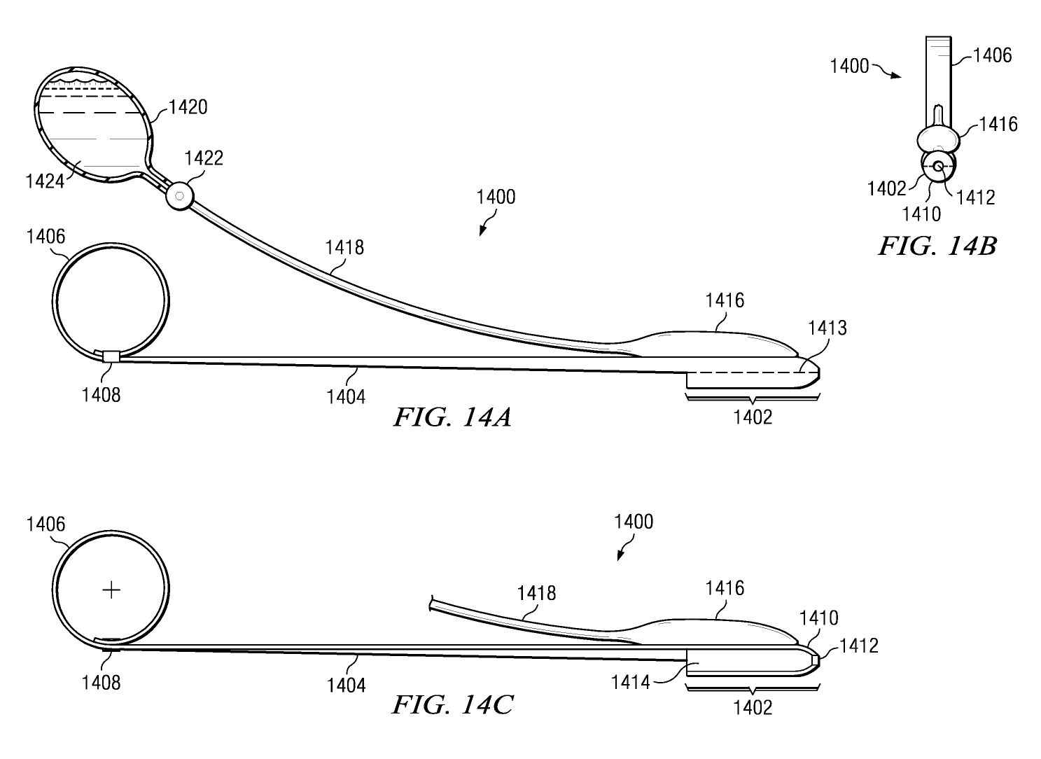

FIGS. 14A and 14B are exterior side and front views of an alternate sheath 1400 for use with a sling assembly such as assembly 100 of FIG. 1. Sheath 1400 includes a cover portion 1402 for enclosing an anchor member (not shown) during installation of the sling assembly. Sheath 1400 further includes a cord portion 1404 having an end formed into a handle 1406 to facilitate removal of the sheath from the anchor. In one embodiment, the rear portion of cord 1404 is looped and secured with a clip 1408 to form circular handle 1406. Cover portion 1402 includes a longitudinally extending cavity 1414 wherein an anchor member is disposed prior to placement of the sling assembly. An aperture 1412 is provided at the end of cover portion 1402 allowing the trochar point (FIG. 1) to protrude from the cover. Cover portion 1402 may include a series of longitudinal scores 1413 that are preferably formed along the length of the cover to assist in removal of the sheath from the anchor after placement.

FIG. 14C is a cross-sectional view of sheath 1400. In one variation, sheath 1400 is formed from tubing having a thermally molded distal end 1410 and wherein cord 1404 and handle 1406 are formed from split portions of the tubing. The distal end 1410 of cover portion 1402 may be formed with a rounded or curved profile to facilitate insertion through the tissues of the patient. In one variation, sheath 1400 may be formed from polypropylene tubing and have dimensions similar to sheath 116 of FIGS. 8A-8C.

In the illustrated embodiment, a dissection balloon 1416 is attached to sheath 1400. In one embodiment, dissection balloon 1416 extends parallel to and along the length of cover portion 1402. Balloon 1416 may be a non-compliant balloon of the type known for use in medical procedures in the fields of angioplasty, orthopedics, and urology. Non-compliant balloons the same and similar to those described in U.S. Pat. No. 6,748,425 to Beckham and co-pending pending publication Nos. US 2006-0085022 A1 to Hayes, US 2006-0085023 A1 to Davies, Jr., and US 2006-0085024 A1 to Pepper, the disclosures of which are hereby incorporated herein by reference, may be suitable for use. Dissection balloon 1416 is shown in FIGS. 14A-14C in an inflated configuration for the purpose of illustration. However, the relative dimensions of balloon 1416 may differ from the illustrated embodiment. Prior to inflation, the balloon 1416 will preferably lie snuggly against the sides of cover 1410 in order to present the smallest possible cross-section.

Dissection balloon 1416 may be attached to sheath 1400 by means of thermal bonding or a suitable medical adhesive. Balloon 1416 may also be mechanically attached to sheath 1400 by means of a film, fiber or fabric (not shown) wrapped around the balloon and the sheath. Balloon 1416 may also be co-extruded with the tubing used to form sheath 1400. Polymers and copolymers that may be used for balloon 1416 include the conventional polymers and copolymers used in medical balloon construction, including, but not limited to, polyethylene, polypropylene, propylene-ethylene copolymers, polyethylene terephthalate, polycaprolactam, polyesters, polyethers, polyamides, polyurethanes, polyimides, ABS, nylons, copolymers, polyester/polyether block copolymers, ionomer resins, liquid crystal polymers, and rigid rod polymers. Balloon 1416 may be formed from multiple layers of such polymers and copolymers, with or without fiber or fabric reinforcement.

Referring again to FIG. 14A, an inflation tube 1418 is connected to an inflation device 1420 for inflating balloon 1416. In the illustrated embodiment, the inflation device is a hand-operated bulb; however, other inflation devices such as those used with medical balloons may be used. In one variation, a valve 1422 is provided for closing tube 1418 to retain pressure in balloon 1416. The balloon 1416 is typically inflated with an incompressible fluid 1424 such as a saline solution. Alternatively, a compressible fluid such as air or nitrogen may be used to inflate balloon 1416. In other embodiments, other fluid compression assemblies such as used in angioplasty or the assembly 1314 of FIG. 13 may be utilized to inflate balloon 1416.

The surgical placement of a flexible sling member utilizing sheath 1400 with dissection balloon 1416 may be the same or similar to the procedure previously described in connection with FIG. 13. As opposed to incising the vaginal wall, a small puncture opening is created using an appropriate surgical instrument. During the procedure, dissection balloon 1416 is inflated with inflation bulb 1420 to create a channel suitable for deployment of the flexible sling. As compared to incising the vaginal wall, the small puncture required for deployment of the sling is anticipated to result in decreased tissue damage, bleeding and post-surgical discomfort.

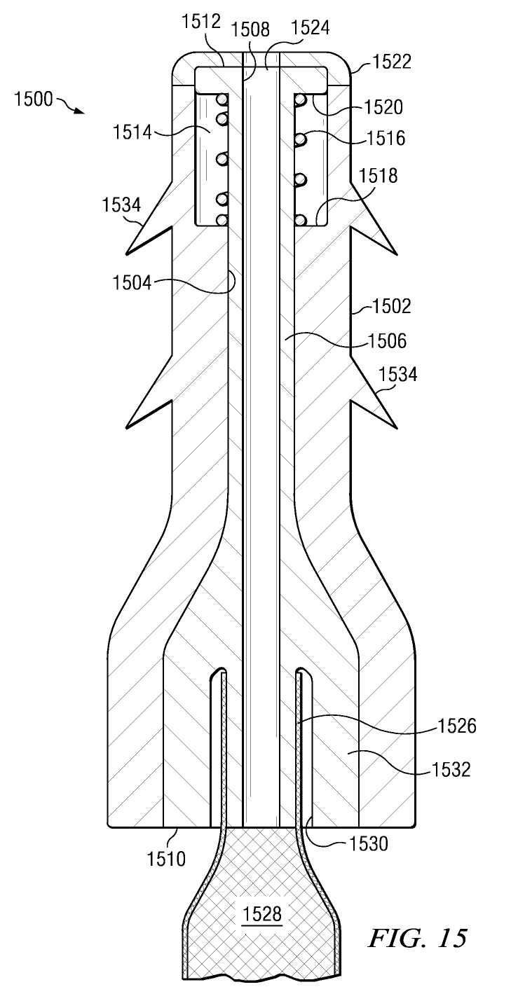

FIG. 15 is a partial cross-sectional view illustrating an alternative anchor body 1500 for use with a sling assembly such as illustrated and described in connection with FIG. 1. As shown, anchor body 1500 includes a generally tubular outer anchor member 1502 that defines a longitudinally extending cavity 1504. An inner tubular member 1506 is slidably disposed in cavity 1504 for limited movement relative to outer tubular member 1502. Inner tubular member 1506 may be fabricated in substantially the same manner as described in connection with FIGS. 5A-5F, with or without folding the end of the sling member. Inner tubular member 1506 includes a needle passage 1508 extending longitudinally through the member from a proximal end 1510 to a distal end 1512 for receiving a stylet therein. A tear-away removable sheath including a cover and a removal cord may be provided as illustrated and described in connection with FIG. 1 or FIG. 14.

The distal end of cavity 1504 includes an enlarged recess 1514 for receiving a spring 1516 between the outer anchor member 1502 and the inner tubular member 1506. As illustrated, spring 1516 is retained between an annular end wall 1518 of recess 1514 and a flange 1520 formed on the distal end 1512 of inner tubular member 1506. Spring 1516 biases inner tubular member 1506 toward the distal end of anchor body 1500 while allowing the inner tubular member to move a limited distance in the proximal direction. In the illustrated embodiment, spring 1516 is a coil spring and may be formed from surgical stainless steel, a plastic or other suitable material. In other variations, different types of springs, such as one or move wave or disk type springs, may be used to bias inner tubular member 1506. In some embodiments, flange 1520 may be an integral portion of member 1506 and in other embodiments, flange 1520 may be a separately-formed element that is attached to member 1506 by means of thermal welding, adhesives, threads or other known connections.

An assembly cap 1522 is attached to the distal end of outer tubular member 1502 to enclose inner tubular member 1506 and spring 1516 in the outer tubular member. Assembly cap 1522 is formed with a central opening 1524 aligned with needle passage 1508 to allow the trochar point of a stylet to extend through anchor body 1500. Assembly cap may 1522 may be thermally or ultrasonically welded to outer tubular member 1502 or attached to the outer tubular member with an appropriate adhesive.

As illustrated, an end 1526 of a sling member 1528 is affixed in an annular slot 1530 formed in an enlarged end portion 1532 of inner tubular member 1506. In one embodiment, enlarged end portion 1532 may have a wall thickness substantially greater than the wall thickness at the distal end 1512 of inner tubular member 1506. As illustrated, annular slot 1530 is concentric with respect to needle passage 1508. In some embodiments, the end of sling member 1528 may be secured to the annular slot 1530 and then folded upon itself to form an extendable adjustment loop as illustrated and described in connection with FIG. 4. In other embodiments, the sling member 1528 may be secured in the slot 1530 and the extendable adjustment loop may be omitted.

Outer tubular member 1502 includes a plurality of proximally-oriented prongs 1534 for securing anchor body 1500 in position. Once a sling assembly utilizing anchor body 1500 is deployed in a patient as described above, prongs 1534 embed in the patient's tissue to secure the end of sling member 1528 in the desired location. Although outer tubular member 1502 is substantially fixed in position by means of prongs 1534, spring 1516 allows inner tubular member 1506 and sling member 1528 to move a limited distance relative to the outer tubular member. It is anticipated that the limited movement of sling member 1528 permitted by the combination of moveable inner tubular member 1506 and spring 1516 will result in reduced patient discomfort as the patient moves about.

It will be appreciated by those skilled in the art having the benefit of this disclosure that these methods and apparatus for pubic sling insertion provide significant advantages in comparison to earlier methods and apparatus. It should be understood that the drawings and detailed description herein are to be regarded in an illustrative rather than a restrictive manner, and are not intended to be limiting to the particular forms and examples disclosed. On the contrary, included are any further modifications, changes, rearrangements, substitutions, alternatives, design choices, and embodiments apparent to those of ordinary skill in the art, without departing from the spirit and scope hereof, as defined by the following claims. Thus, it is intended that the following claims be interpreted to embrace all such further modifications, changes, rearrangements, substitutions, alternatives, design choices, and embodiments.

What is claimed is:

1. An adjustable-length support sling assembly for internal placement within a patient, the support sling assembly comprising:
an elongated, generally flat sling member formed of a mesh material having a first end and a second end;
a first anchor member disposed at a first end of the sling member and a second anchor member disposed at a second end of the sling member;
each respective anchor member having a generally tubular wall defining a needle passageway extending longitudinally therethrough from a proximal end to a distal end;
the wall of each respective anchor member defining a plurality of proximally-directed prongs projecting from the outer surface thereof and an enlarged collar portion at the proximal end thereof, the collar portion having a wall thickness that is substantially greater than the wall thickness at the distal end;
the collar portion of each respective anchor member further defining an annular slot formed in the proximal end of the anchor member and extending longitudinally toward the distal end, the annular slot being concentrically disposed with respect to the needle passageway;
each respective end of the sling member being permanently affixed to the corresponding anchor member, and a respective further portion of the sling member adjacent each respective end being folded upon itself by a predetermined length and releasably secured within the annular slot of the corresponding anchor member;
the dimensions of each respective annular slot being selected to retain the corresponding folded portion of the sling member within the slot until the tension applied by the sling member exceeds a respective predetermined tension level; and
whereby the anchor members are securable in the tissue of a patient by means of the proximally-directed prongs, each respective anchor member will maintain tension on the sling member up to the corresponding predetermined tension level, and each respective anchor member will release the corresponding predetermined length of sling member from the corresponding annular slot when the tension applied by the sling member exceeds the corresponding predetermined tension level.

2. A support sling assembly in accordance with claim 1, wherein the mesh material of the sling member is a woven material having a thickness less than about 0.015 inches.

3. A support sling assembly in accordance with claim 1, wherein the mesh material of the sling member is a unitary sheet having holes formed by perforation.

4. A support sling assembly in accordance with claim 1, wherein the mesh material of the sling member is polypropylene.

5. A support sling assembly in accordance with claim 1, further comprising a removable sheath for each of the anchor members, each of the removable sheaths including a cover portion and a cord portion and wherein the cover portion is disposed over each respective anchor member and covers the plurality of proximally-directed prongs projecting from the anchor members to facilitate placement of the anchor member in a patient.

6. A support sling assembly in accordance with claim 5, wherein the cover portion of the removable sheath includes a least one score to facilitate removal of the sheath after the anchor member is placed in a patient.

7. A support sling assembly in accordance with claim 5, wherein at least one of the removable sheaths further comprises a non-compliant medical balloon attached to the sheath.

8. A support sling assembly in accordance with claim 7, wherein the non-compliant medical balloon is attached to an exterior surface of the cover portion of the removable sheath.

9. A surgical method for treating female urinary incontinence, the method comprising:
making an incision in the vaginal wall;
performing para-urethral dissection through the incision toward the minor obturator muscles;
inserting a stylet carrying a first end of an adjustable sling assembly through the incision and laterally through the dissected tissue into a first of the minor obturator muscles;
the first end of the adjustable sling assembly including a first anchor member attached to a first end of a flexible sling member;
removing a protective sheath from the first anchor member while maintaining its position in the first of the minor obturator muscles so as to expose rearward facing prongs on the surface of the anchor;
withdrawing the stylet from the incision, leaving the first anchor member embedded in the first minor obturator muscle;
routing the flexible sling member across the neck of the urethra;
inserting a stylet carrying a second end of the adjustable sling assembly through the incision and laterally through the dissected tissue into a second of the minor obturator muscles;
the second end of the adjustable sling assembly including a second anchor member attached to a second end of the flexible sling member;
removing a protective sheath from the second anchor member while maintaining its position in the second of the minor obturator muscles so as to expose rearward facing prongs; and
withdrawing the stylet from the incision, leaving the second anchor member embedded in the second minor obturator muscle and thereby creating a tension in the flexible sling member;
closing the incision.

10. The method of claim 9, wherein after closing the incision, the tension of the flexible sling member may be non-surgically adjusted by inserting a dilator into the external urethral opening until it is disposed in the urethra adjacent the internally positioned flexible sling, then pressing the urethra with the dilator against the sling to increase the tension in the sling above a predetermined level, whereupon a folded portion of the flexible sling in at least one of the anchor members is unfolded to increase the length of the flexible sling.

11. A surgical apparatus for positioning a pubic sling in the endopelvic region, comprising:
an elongated stylet having a distal end, a proximal end and a balloon port therebetween, the distal end forming a trochar point, a portion of the stylet between the balloon port and the proximal end being hollow;
a handle mounted to the proximal end of the stylet;
a non-compliant surgical balloon concentrically mounted over the stylet such that the balloon port is in communication with the interior of the balloon;
a fluid reservoir operatively connected to the interior of the balloon via the hollow portion of the stylet and the balloon port;
a fluid compression assembly that selectively compresses the fluid in the reservoir;
wherein the fluid compression assembly can alternately fill and drain the balloon to selectively inflate and deflate the balloon to dissect tissue of the endopelvic region.

12. A surgical apparatus in accordance with claim 11, wherein the fluid reservoir is located in the handle.

13. A surgical apparatus in accordance with claim 11, further comprising a fluid valve mounted on the fluid path between the balloon and the fluid reservoir for selectively restricting the flow of fluid into and out of the balloon.

14. An anchor for a support sling assembly for internal placement within a patient, the support sling assembly including an elongated, generally flat sling member formed of a mesh material having a first end and a second end, the anchor comprising:
a generally tubular outer member including a plurality of proximally-directed prongs projecting from an outer surface thereof, the outer tubular member defining a longitudinal cavity extending through the outer tubular member;
a generally tubular inner member slidably disposed within the longitudinal cavity of the tubular outer member for movement over a limited distance relative to the tubular outer member, the tubular inner member defining a needle passageway extending longitudinally therethrough from a proximal end to a distal end;
a spring member disposed between the tubular outer member and the tubular inner member, the spring member biasing the tubular inner member relative to the tubular outer member such that the tubular inner member can move a limited distance relative to the tubular outer member;
wherein an end of the sling member may be permanently affixed to the tubular inner member of the anchor such that the inner tubular member may move a limited distance relative to the outer tubular member when the anchor is secured in the tissue of a patient by means of the proximally-directed prongs.

15. An anchor in accordance with claim 14, wherein the tubular inner member further comprises an enlarged end portion and wherein the tubular inner member further defines an annular slot in the enlarged end portion for receiving an end of the sling member therein.

16. An anchor in accordance with claim 15, wherein the annular slot is concentrically disposed with respect to the needle passageway.

17. An anchor in accordance with claim 14, wherein the tubular outer member further defines an annular recess and wherein the spring is disposed in the annular recess between the tubular outer member and the tubular inner member.

18. An anchor in accordance with claim 17, wherein the tubular inner member further comprises a flanged end and wherein the tubular outer member further defines an annular recess having an annular end wall and wherein the spring is disposed in the recess between the flange and the annular end wall.

19. An anchor in accordance with claim 14, further comprising a removable sheath having a cover portion and a cord portion and wherein the cover portion is disposed over the anchor and covers the plurality of proximally-directed prongs projecting from the anchor members to facilitate placement of the anchor member in a patient.

20. An anchor in accordance with claim 19, further comprising an elongated non-compliant medical balloon attached to an exterior portion of the cover portion of the removable sheath.
